(19) United States
(12) Patent Application Publication
(10) Pub. No.: US 2006/0091033 A1
(43) Pub. Date: May 4, 2006

(54) Arrangement and method for preserving the new-boneforming effect of growth-stimulating substances for an implant product

(75) Inventor: Jan Hall, Goteborg (SE)

Correspondence Address: CONNOLLY BOVE LODGE & HUTZ LLP, SUITE 800, 1990 M STREET NW, WASHINGTON, DC, 20036-3425, US

(73) Assignee: NOBEL BIOCARE AB, GOTEBORG (SE)

(21) Appl. No.: 10/520,009

(22) PCT Filed: Jun. 18, 2003
(86) PCT No.: PCT/SE03/01021

§ 371 Date: Sep. 15, 2005

(51) Int. Cl. A61B 19/02; B65B 31/02

(52) U.S. Cl. 206/438; 206/524.8; 206/524.4; 53/432; 53/467

(57) ABSTRACT

In an arrangement and method, use is made of a container for preserving, even for a long time, the new-bone-forming effect of growth-stimulating substances (GS) applied to one or more implant products. The container is arranged so as, dependent on being acted upon, to allow accessibility of the product concerned with applied GS as the time of its use. The container is arranged to enclose the product or the products with applied GS in an environment which is essentially free from air, water and moisture. The container functions as a vacuum container, and the storage times for GS can be extended considerably in relation to previously used arrangements and methods.

The present invention relates to an arrangement for a container for preserving, even for a long time, the new-bone-forming effect of growth-stimulating substances (GS) applied to one or more implant products. In this connection, the container is arranged so as, dependent on being acted upon, to allow accessibility of the product concerned with applied GS at the time of use of the product. Here, long time means, for example, periods of several years.

It has previously been proposed that a GS which is intended to be used on implants, for example dental implants, should be kept in a frozen environment until use on the spot by a surgeon, dentist etc. The user in question should then mix the frozen molecules with an aqueous solution in order to achieve an appropriate concentration which is to be applied to the product/the implant in question at the point of use. It has also been proposed that the molecules be applied to the product and that the combination of molecules/product be frozen during storage and transport.

Achieving the correct concentration and correct application with the correct application technique on the product in question in order to arrive at a desired result with regard to new-bone growth in the dental context is difficult and requires extensive experience and knowledge. In order to avoid incorrect use sur place, it has become desirable for the concentration and application techniques to be performed in the factory and for the product provided with GS to be supplied in finished state to the location where the dental work is to be carried out. In this connection, the need exists to manufacture products with GS in the factory and to effect interim storage of the products in connection with sale and distribution to the users. The object of the present invention is to resolve inter alia this problematic situation and to start from the knowledge the storage time for the GS applied to the product is highly dependent on whether or not the GS is exposed to air, water or a moist environment. In this respect, the greater the exposure of the GS to air, water and moisture, the shorter will be the storage time in order for effective use of the product in question with GS to be possible. The invention also aims to solve this problem by making possible long storage times in this connection, that is to say storage times of several years.

An arrangement according to the invention can be considered to be characterized mainly in that the container is arranged to enclose the product or the products with applied GS in an environment which is essentially free from air, water and moisture.

In this connection, the container can be in the form of a glass ampoule, or be made of metal which makes the environment free from air, water and moisture possible, for example metal in the form of titanium, stainless steel etc. The container can consist of a vacuum container which has an internal pressure for the product or the products with applied GS of <10⁻⁴ mbar. Said environment can comprise one or more essentially inert gases, for example argon, nitrogen or helium, which in one embodiment are used in connection with evacuation of the container in question.

A method according to the invention can be considered to be characterized mainly in that the container is made to enclose the product or the products with applied GS in an environment which is essentially free from air, water and moisture. In one embodiment, the product with associated GS can, be applied in a glass tube provided with a bottom, the interior of which is connected to a vacuum pump. After evacuation, a burning means employed can be activated for formation of a closed glass ampoule by sealing the glass tube part enclosing the product with GS. In the case of metal, a first part or lower part made of foil-shaped metal, for example titanium or stainless steel, can be used. The product is arranged on the first part with associated GS applied. A second part or upper part likewise made of foil-shaped metal is applied to the first part or lower part with the product or the products enclosed. The space between the parts which is intended for the product or the products is evacuated and/or filled with gas and sealed by means of laser welding or what may be referred to as extended single-spot welding, that is to say welding by means of one stroke along the entire sealing length. In some applications, seaming can also be employed as the sealing method. Other developments of the method emerge from the subsequent subclaims for the method.

By virtue of what is proposed above, accurate production of optimum products loaded or provided with GS can take place in the factory. Even after a long storage time, the product with GS can give optimum results with regard to new-bone growth in various dental situations. As examples of GS, mention may be made of matrix molecules, growth and differentiation factors and peptides with growth-stimulating properties. GS can also be applied to substances such as autologous bone, allogenic bone or synthetic preparations, these substances and preparations also then having a long storage time with the principle employed. When air containing water and moisture is replaced by inert gas free from water or moisture or evacuation of the container concerned is effected, known equipment can be used for the gas supply and evacuation.

A for the present proposed arrangement and method based on the characteristics significant of the invention will be described below with simultaneous reference to accompanying drawings in which

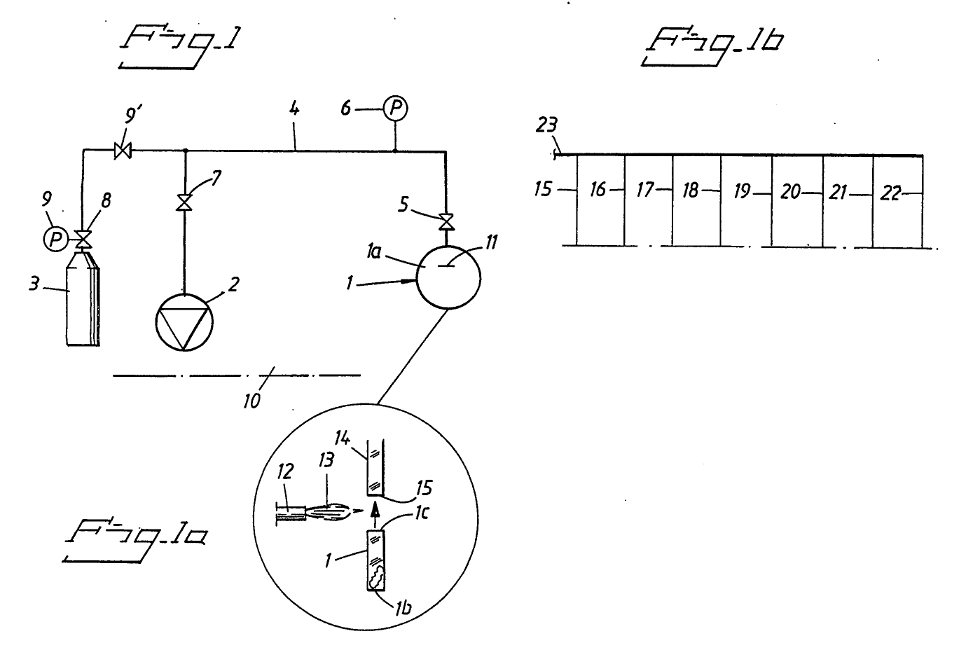

FIG. 1 shows in the form of a schematic diagram the formation of a glass ampoule with a product provided with GS introduced, the interior of the glass ampoule being connectable to on the one hand a vacuum pump and on the other hand a gas bottle;

FIG. 1a shows in principle the sealing function for the glass ampoule;

FIG. 1b shows in principle a pumping arrangement for a number of ampoules;

FIG. 2 shows a horizontal view of a first part or lower part made of metal foil with a compartment for an implant or another product with applied GS;

FIG. 3 shows a vertical section of the first part or lower part according to FIG. 2;

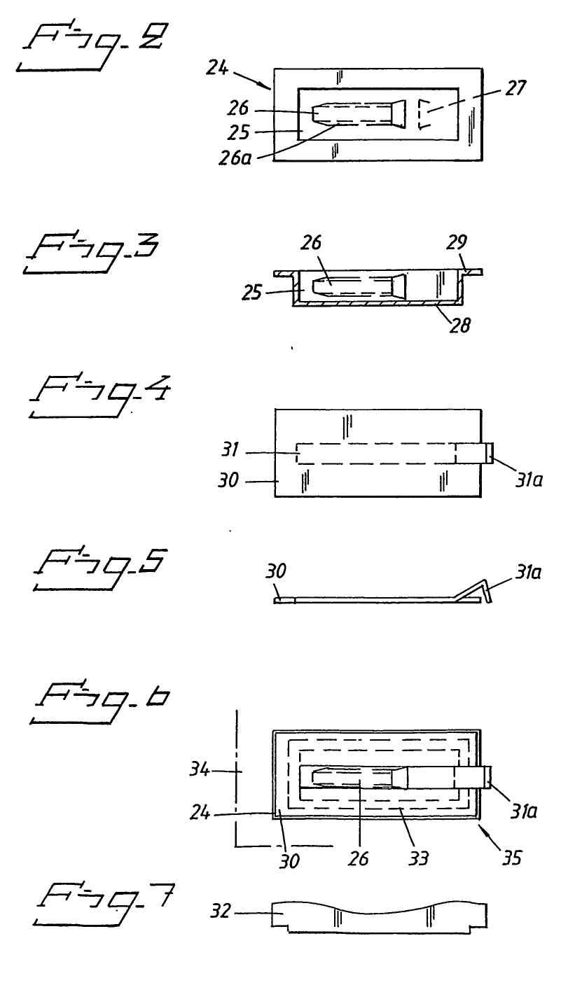

FIG. 4 shows a horizontal view from above of a second part or upper part intended to be applied to the first part or lower part and provided with a tear-off strip for uncovering the product with applied GS enclosed by the first and second parts;

FIG. 5 shows a side view of the upper part according to FIG. 4;

FIG. 6 shows a horizontal view of the first and second parts in an a joined-together state, the parts having been joined together by means of a welding procedure, and

FIG. 7 shows a side view of a part of the welding tool for extended single-spot welding which brings about the mutual sealing of the first and second parts with the implant with GS located inside.

In FIG. 1, what will become a glass ampoule is indicated symbolically by 1. A vacuum pump of a kind known per se is shown by 2 and a gas container by 3. The ampoule is connected to a line system 4 which is indicated schematically. The connection of the interior 1a of the glass ampoule to the line system is effected by means of melting the glass material. Also connected to the system is a manometer 6 in order to make it possible to establish an appropriate pressure in the system. The pump 2 is connected to the line system via valve means 7. The gas bottle 3 is provided with a regulating valve 8, to which a manometer, here called the second manometer 9, can be connected. The gas bottle arrangement can be connected to the line system 4 via a second valve means 9′. A product indicated schematically, for example an implant, which has the reference number 11, is introduced into the glass ampoule. The product is provided with GS in the manner indicated in the applications filed by the same applicant and inventor. In this connection, reference may be made to patent applications WO 00/72775 and WO 00/72777 by the same applicant as in the present patent application. Reference may also be made to the article “Properties of a New Porous Oxide Surface on Titanium Implants, Volume 1: The Oxidized Titanium Surface, Applied Osseointegration Research”, October 2000, published by among others the inventor according to the present patent application.

A technique known per se can be used for sealing the ampoule 1. By means of the vacuum pump, the interior of the ampoule is evacuated to a value which is <10⁻⁴ in the illustrative embodiment. In the space 10, the glass ampoule thus evacuated is sealed with the aid of a rotating burner 12 according to FIG. 1. The burner works with a gas flame 13. The glass ampoule 1 is provided with a sealed bottom part 1b, and the top part 1c is also sealed by means of the gas flame 13. As the glass ampoule is provided with an indication, it can then be opened by breaking. In some cases, it is desirable for the glass ampoule to be provided with a positive pressure by an inert gas, for example argon, in order to prevent glass fragments entering the ampoule when it is broken. The ampoule is then filled with gas from the gas bottle 3 which is connected via the regulating valve 8 and the valve means 9 to the line system and via the connection valve 5.

FIG. 1 shows a multiple arrangement for a number of glass ampoules. Each glass ampoule 15, 16, 17, 18, 19, 20, 21 and 22 is connected internally to a line 23, which can in turn be connected in a manner known per se to said gas bottle 3 and the vacuum pump. Products provided with GS can be introduced into the ampoules, after which the latter can be evacuated, filled with gas and sealed in accordance with the above.

FIGS. 2-7 show the case when the container consists of two foil-shaped metal parts which can be welded together with one another with the product with GS located inside during simultaneous evacuation of the space for the product in connection with evacuation. FIG. 2 shows a first part or lower part 24 with a space 25 for a product in the form of an implant 26 which has been coated with GS on its external-thread 26a in the way indicated above. The space 25 can contain more than one product, which has been symbolized by 27.

FIG. 3 shows the shape of the first part or lower part from the side; the part can be regarded as comprising a space part 28 and a flange part 29.

FIG. 4 shows a second part or upper part which is intended to be joined together with the part according to FIGS. 2 and 3. The second part or upper part is indicated by 30 and is provided with a tear-off strip 31. The second part or upper part is arranged in a vacuum-tight manner in relation to the lower part by means of welding around the flange part, for example laser welding. The tear-off strip has at one of its ends a tab 31a, by means of which it is possible to take hold of the strip and tear the same off from the part 30 in question.

In accordance with FIG. 5, the tab part 31a has a shape which is easy to grip and facilitates tearing-off of the flap 31 from the part 30.

FIG. 6 shows the case where the second part 30 has been applied to the first part 24. Sealing is effected by means of laser welding or the welding stamp 32 shown symbolically in FIG. 7 which is arranged to establish a vacuum-tight seal 33 between the parts 24 and 30 by interaction. Sealing is effected at the flange 29 shown in FIG. 3. Sealing is effected by a single interaction between the stamp and the parts 30 and 40, and the welding function is referred to here as extended single-spot welding. Stamping is preceded by evacuation and in some cases by a subsequent gas-filling of the inner space (compare 25 in FIG. 3) of the parts, which evacuation and gas-filling are performed in a symbolically indicated apparatus 34 of a type known per se. Reference is also made to the arrangement above according to FIGS. 1-1b. The weld 33 runs all the way round and on the whole follows the center line of the flange 29. The container 35 thus evacuated makes possible long-term storage of the product 26 with applied GS for said long time without the GS degenerating with regard to its new-bone-forming function.

The invention is not limited to the embodiment shown above by way of example but can undergo modifications within the scope of the following patent claims and the inventive idea.

1. An arrangement for a container for preserving the new-bone-forming effect of growth-stimulating substances (GS) applied to at least one implant product, the container being arranged so as, dependent on being acted upon, to allow access to the at least one implant product with applied GS at the time of use of the at least one implant product with applied GS, wherein the container is arranged to enclose the at least one implant product with applied GS in an environment which is essentially free from air, water and moisture.

2. The arrangement of claim 1, wherein the container is in the form of a glass ampoule.

3. The arrangement of claim 1, wherein the container is made of metal which makes the environment free from air, water and moisture possible.

4. The arrangement of claim 1, wherein the container has been evacuated to an internal pressure less than 1 mbar.

5. The arrangement of claim 1, wherein said environment comprises at least one essentially inert gases free from air, water and moisture.

6. A method for a container for preserving the new-bone-forming effect of growth-stimulating substances (GS) applied to at least one implant product, comprising:
enclosing the at least one implant product with applied GS in a container that provides an environment which is essentially free from air, water and moisture, wherein the container is arranged to be openable, dependent on being acted upon, so as to allow access to the at least one implant product with applied GS at the time of use of the at least one implant product.

7. The method of claim 6, wherein the container is made as a glass ampoule or of metal capable of preserving the environment free from air, water and moisture.

8. The method of claim 6, wherein the container is evacuated with an internal pressure of less than 1 mbar.

9. The method of claim 6, wherein the environment free from air, water and moisture is formed by means of one or more gases free from water and moisture.

10. The method of claim 6, wherein the at least one implant product with applied GS is introduced into an open glass ampoule, the interior of which is connected to a vacuum pump, and wherein, when a low internal vacuum pressure has been reached, the glass ampoule is sealed using a burning means.

11. The method of claim 10, wherein the at least one implant product with applied GS is introduced into a glass tube provided with a bottom, wherein the interior of the glass tube is connected to the vacuum pump, and wherein the burning means is activated for formation of a closed glass ampoule by sealing a glass tube part enclosing the at least one implant product with applied GS.

12. The method of claim 10, wherein the interior of the ampoule or of the glass tube part is connected temporarily to a gas container.

13. The method of claim 6, wherein the at least one implant product with applied GS is arranged in a first part made of foil-shaped metal, wherein a second part made of foil-shaped metal is applied to the first part and over the at least one implant product, and wherein a space between the first and second parts which is intended for the at least one implant product or the with applied GS is evacuated and/or filled with gas and sealed by means of extended single-spot welding or laser welding.

14. The method of claim 13, wherein at least one of the first part and the second part is made with a tear-off foil strip for access to the at least one implant product with applied GS at the time of use.

15. The arrangement of claim 3, wherein the metal is titanium or stainless steel.

16. The arrangement of claim 1, wherein the container has been evacuated to an internal pressure of less than 10⁻³ mbar.

17. The arrangement of claim 5, wherein the at least one essentially inert gas is argon.

18. The arrangement of claim 7, wherein the metal is titanium or stainless steel.

19. The arrangement of claim 6, wherein the container is evacuated with an internal pressure of less than 10⁻³ mbar.

20. The method of claim 10, wherein the burning means is a rotating burner.

21. The method of claim 13, wherein the first part is a lower part and the second part is an upper part.
